(19) United States
(12) Patent Application Publication
(10) Pub. No.: US 2014/0182595 A1
(43) Pub. Date: Jul. 3, 2014

(54) Tracheostomy Tube with Cannula Connector

(71) Applicant: COVIDIEN LP, Mansfield, MA (US)

(72) Inventors: Seamus Maguire, Athlone (IE); Stanley Brian Kaus, Longmont, CO (US); Mark Raymond Behlmaier, Erie, CO (US)

(73) Assignee: Covidien LP, Mansfield, MA (US)

(21) Appl. No.: 13/729,839

(22) Filed: Dec. 28, 2012

(51) Int. Cl. A61M 16/04

(52) U.S. Cl. CPC A61M 16/0465; A61M 16/0434
USPC 128/207.15; 128/207.14; 156/242

(57) ABSTRACT

A tracheal tube assembly includes a cannula connector that includes a cannula connector cap coupled to a flange assembly. The flange assembly is in turn coupled to a cannula. The flange assembly includes a portion that extends through slots formed at or near a proximal end of the cannula connector cap. The flange assembly may be overmolded.

### BACKGROUND

The present disclosure relates generally to the field of tracheal tubes and, more particularly, to a tracheal tube including a cannula with a connector end.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of situations exist in which artificial ventilation of a patient may be desired. For short-term ventilation or during certain surgical procedures, endotracheal tubes may be inserted through the mouth to provide oxygen and other gasses to a patient. For other applications, particularly when longer-term intubation is anticipated, tracheostomy tubes may be preferred. Tracheostomy tubes are typically inserted through an incision made in the neck of the patient and through the trachea. A resulting stoma is formed between the tracheal rings below the vocal chords. The tracheostomy tube is then inserted through the opening. In general, two procedures are common for insertion of tracheostomy tubes, including a surgical procedure and a percutaneous technique.

Such tubes may include an inflatable balloon cuff, or may be cuffless. In both cases, a connector is typically provided at an upper or proximal end where the tube exits the patient airway. Standard connectors have been developed to allow the tube to then be coupled to mechanical ventilation equipment to supply the desired air or gas mixture to the patient, and to evacuate gasses from the lungs.

Current designs for such tubes may allow for easy connection to an upper connector, but may have various structures, some quite complex, for conveying air between the connector and a cannula that extends into the patient. In some cases, a soft plastic or rubber is used for the connector, providing a flexible seal with the interfacing ventilation assembly, although such soft materials may collapse or deform when pressed into the mating connector element. However, more rigid connector materials may also be associated with difficulties. For example, rigid connectors may deform a cannula during a coupling step, which in turn may decrease the size of the ventilation path.

There is a need, therefore, for improved tracheal tubes, and particularly for improved tracheostomy tubes. It would be desirable to provide a tube that allows for a robust connector that also maintains a suitable airway diameter when coupled to upstream or downstream tubing.

### BRIEF DESCRIPTION

This disclosure provides a novel tracheal tube designed to respond to such needs with a cannula connector that couples to a cannula with reduced deformation of the coupling end of the cannula. The cannula connector includes a connector cap component and a flange assembly component. In certain embodiments, the flange assembly is formed from a relatively soft material while the connector cap component is formed from a relatively rigid component. The flange assembly is coupled to and extends within the connector cap to form the interior passageway of the connector. In this manner, the interior passageway may be formed from a relatively soft or compliant material while maintaining exterior rigidity for robust connection with ventilation tubing. Further, the interior material, which may be different than the cap material, may allow flexibility in coupling techniques to the cannula. For example, in one embodiment, the cannula is coupled to the material from which the flange assembly is formed via bonding or adhesion to the flange assembly portion disposed in the interior passageway. The material of the flange assembly may be selected to facilitate a desired coupling technique. In one embodiment, the coupling may be performed in conjunction with techniques for widening or maintaining the interior diameter of the proximal end of the cannula during a coupling process. Further, the flange assembly may be molded to and/or over the connector cap to enhance the strength of the coupling of the connector cap to the flange assembly. In one embodiment, the connector cap may include passageways or slots configured to allow the material of the flange assembly to pass through portions of the connector cap.

Thus, in accordance with a first aspect, a tracheal tube assembly includes a cannula configured to be positioned in a patient airway. The assembly further includes a cannula connector comprising: a cannula connector cap comprising a plurality of slots spaced about the cannula connector cap at or near a proximal end of the cannula connector cap; and a flange assembly coupled to the cannula connector cap and the cannula and comprising a flange, wherein a portion of the flange assembly extends through the slots

In accordance with another aspect, a method of manufacturing a tracheal tube assembly includes providing a cannula connector cap comprising a plurality of slots spaced about a circumference of the cannula connector cap at or near a proximal end of the cannula connector cap; molding a flange assembly to form a flange coupled to the cannula connector cap, wherein molding the flange assembly comprises molding over at least a portion of the cannula connector cap such that a portion of the flange assembly extends through the slots and wherein the flange assembly is disposed inside of the cannula connector cap to define an interior passageway; and coupling a proximal end of a cannula to the interior passageway.

Also disclosed herein is a tracheal tube assembly that includes a cannula configured to be positioned in a patient airway; an inflatable cuff disposed on the cannula; a cannula connector cap; a flange assembly molded over a portion of the cannula connector cap to form an interior passageway of the cannula connector cap and coupled to the cannula; an inflation line in fluid communication with the inflatable cuff and extending through the flange assembly and the cannula connector cap, wherein the inflation line terminates in a pilot balloon assembly and wherein the pilot balloon assembly comprises opposing wings that surround a valve configured to receive an inflation device.

### BRIEF DESCRIPTION OF THE DRAWINGS

Various aspects of the disclosed techniques may become apparent upon reading the following detailed description and upon reference to the drawings in which:

FIG. 1 is a perspective view of a tracheal tube with a cannula connector in accordance with embodiments of the present disclosure;

FIG. 2 is an exploded view of the tracheal tube of FIG. 1;

FIG. 3 is perspective view of a cannula connector cap;

FIG. 4 is a cross-sectional view of the cannula connector cap of FIG. 3;

FIG. 5 is a top view of the tracheal tube of FIG. 1;

FIG. 6 is a cross-sectional view of a tracheal tube in accordance with embodiments of the present disclosure;

FIG. 7 is a detail view of an inflation line syringe connector; and

FIG. 8 is a perspective view of an alternative cuffless tracheal tube in accordance with embodiments of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The tracheal tubes are capable of providing mechanical ventilation to the lungs, and capable of supporting all other functions of standard tracheal tubes (e.g. sealing, positive pressure generation, suctioning, irrigation, drug instillation, etc). The tracheal tubes can be used in conjunction with all acceptable auxiliary airway devices (e.g. heat and humidity conservers, mechanical ventilators, humidifiers, closed suction systems, capnometers, oxygen analyzers, mass spectrometers, PEEP/CPAP devices, etc). Furthermore, although the embodiments of the present disclosure illustrated and described herein are discussed in the context of tracheal tubes such as tracheostomy tubes, it should be noted that presently contemplated embodiments may include a tracheal tube assembly used in conjunction with other types of airway devices. For example, the disclosed embodiments may be used in conjunction with a single-lumen tube, an endotracheal tube, a double-lumen tube (e.g., a Broncho-Cath™ tube), a specialty tube, or any other airway device with a main ventilation lumen. As used herein, the term “tracheal tube” may include an endotracheal tube, a tracheostomy tube, a double-lumen tube, a bronchoblocking tube, a specialty tube, or any other airway device.

Further, while the disclosed embodiments are shown with a single cannula (e.g., an outer cannula), it should be understood that any of the disclosed embodiment may be used in conjunction with a disposable or reusable inner cannula. The inner cannula may be inserted into the cannula connector to be placed inside of an outer cannula.

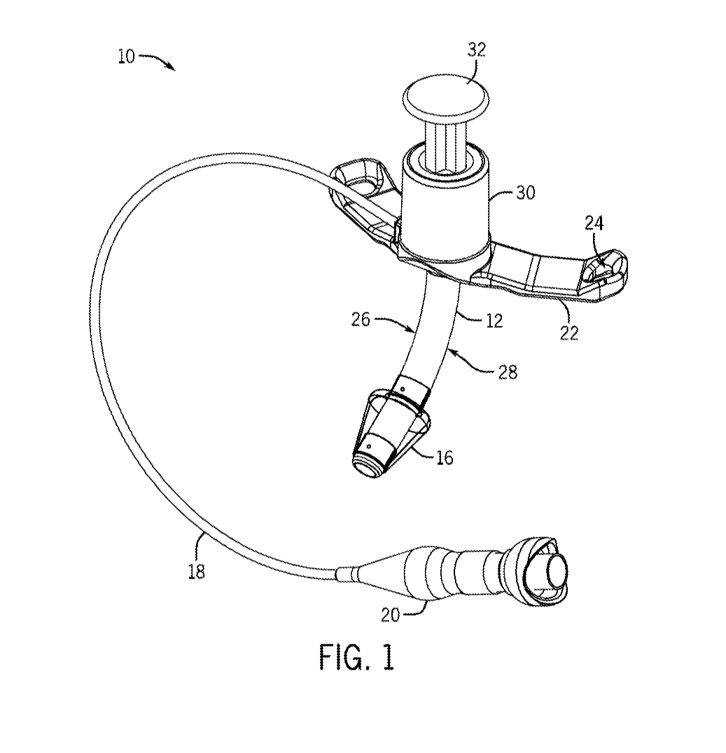

Turning now to the drawings, FIG. 1 is a perspective view of an exemplary tracheal tube 10 in accordance with aspects of the present disclosure. The tracheal tube assembly 10 represented in the figures is a tracheostomy tube, although aspects of this disclosure could be applied to other tracheal tube structures, such as endotracheal tubes. The tracheal tube 10 includes a cannula 12 that defines a ventilation lumen and that facilitates the transfer of gases to and from the lungs. The tracheal tube 10 includes an inflatable cuff 16 disposed on the outer cannula 12. However, certain embodiments of the disclosure may be used in conjunction with cuffless tubes. A proximal end of the tracheal tube 12 may connect to upstream airway devices (e.g., a ventilator) via the appropriate medical tubing and/or connectors. In embodiments that include a cuff 16, an inflation line 18 and pilot balloon assembly 20 is coupled to the cuff 16.

The cannula 12 is illustrated extending from a flange 22. A pair of extending arm portions of the flange 22 extend laterally and serve to allow a strap or retaining member (not shown) to hold the tube assembly 10 in place on the patient. In one embodiment, apertures 24 formed in each side of the flange 22 allow the passage of such a retaining device. In many applications, the flange 22 may be taped or sutured in place as well. During intubation, the tracheal tube assembly 10 is placed through an opening formed in the neck and trachea of a patient and extending into the patient airway. In certain embodiments, the tracheal tube assembly 10 is curved to accommodate the curved tracheal passageway. For example, the outer cannula 12 may be curved in an unbiased state (i.e., outside the patient) such that an inner curve 26 is generally positioned on a ventral side of the patient while the outer curve 28 is positioned on the dorsal side of the patient when the tracheal tube assembly 10 is inserted in the patient. Further, while a distal portion of the outer cannula 12 is inserted within the patient, a proximal portion of the outer cannula 12 couples to a flange assembly including the flange 22 that forms part of the cannula connector 30. It is envisioned that the tracheal tube assembly 10 as provided herein may be provided as an assembly and/or as a kit. The kit may also include a neck strap for retaining the tracheal tube assembly 10 in place. The kit may also include an obturator 32. The tube assembly 10 components (e.g., cannula 12, flange 22, cannula connector 30, cuff 16, and inflation line 18 and pilot balloon assembly 20) may be assembled in the kit.

The tracheal tube 10 includes features that promote secure coupling of the tube components to one another as well as a secure coupling to upstream medical devices. In contrast to tracheal tube assemblies that include mechanical couplings of the components, such as a flange coupled to a connector via a pin or snap connection, the disclosed tracheal tubes 10 feature components that are coupled by molding, overmolding, and/or bonding techniques. In this manner, tracheal tube components that are formed from different materials and that have different material properties may be securely coupled to one another. Further, by providing a rigid exterior surface and tip along with a relatively softer interior, the tracheal tubes 10 as provided combine stability without compressing or deforming the coupling of the cannula 12 to the cannula connector 30. That is, the cannula 12 may be inserted into a soft interior and bonded or otherwise adhered without being pinched at the point of coupling.

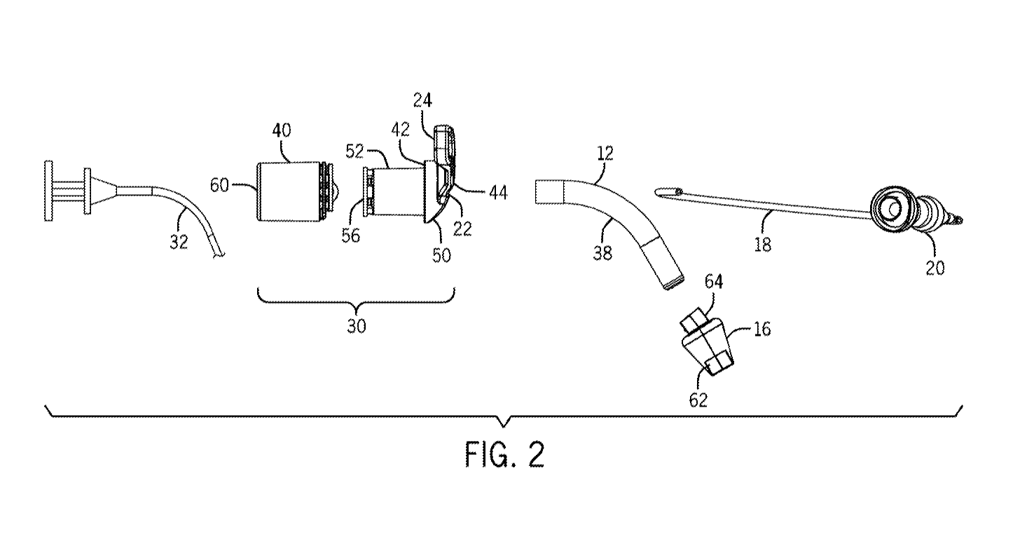

FIG. 2 is an exploded view of the tracheal tube assembly 10. The obturator 32 is typically used during insertion and fits within the cannula connector 30 and the cannula 12 and is subsequently removed to permit flow of ventilation gases. The obturator 32 may also be used as an introducer to allow the tracheal tube 10 to be used for placing the tube 10. As shown, the obturator may protrude past the distal tip 38 of the tube. The obturator 32 may be used to improve insertion and rigidity during the insertion process. The obturator 32 may be sized according to the size of tracheal tube 10. In a particular embodiment, the obturator is manufactured from a polypropylene material.

The cannula connector 30 includes a connector cap 40 and a flange assembly 42. The connector cap 40 forms part of an exterior portion of the cannula connector 30. The flange assembly 42 includes the flange 22 with extending arm portions 44 and apertures 24 configured to hold a neck strap or other retaining device to fit about the patient's neck or head. The extending arm portions 44 extend laterally from a base 50 of the flange assembly 42. The flange assembly also includes an interior portion 52 that is located within an interior of the connector cap 40 such that the interior portion 52 is coaxial with the connector cap 40. The interior portion 52 may be configured to terminate at a proximal end 56 that aligns with a proximal end 60 of the connector cap 40 when assembled. In certain embodiments, the interior portion may completely coat or cover an interior of the connector cap 40 to define an inner passageway for the cannula connector 30. The connector cap 40 may be formed from a material that is relatively more rigid than the flange assembly 42. For example, the connector cap 40 may be formed from a relatively rigid ABS while the flange assembly is formed from PVC, 75 Shore A.

The flange assembly 42 is in turn coupled to the cannula 12, which in particular embodiments is formed from a PVC material such as PVC 85 Shore A. In a specific embodiment, the cannula 12 is phthalate free. The cannula 12 may be formed in various sizes with different interior diameters and/or lengths. The cannula 12 may include an X-ray-visible element (e.g., barium sulphate) within the material to render the cannula 12 visible under X-Ray radiation. In certain embodiments, the distal tip 38 is formed, e.g., melted, to remove sharp edges and to seal a secondary inflation lumen. The cannula 12 may go through additional process of notching to allow the inflation line 18 to be attached through flange and a distal notch may be formed to allow the cuff 16 to be inflated or deflated.

The cuff 16 may be formed of suitable materials to facilitate inflation and formation of a tracheal seal. For example, in one embodiment, the cuff 16 is a thin walled transparent PVC cuff. The cuff 16 may be barrel-shaped or may form a tapered shape. Further, the cuff diameter may be sized to correspond to the cannula size. In one embodiment, a relatively thinner wall cuff 16 provides a low profile when the cuff 16 is deflated, which in turn facilitates intubation and extubation with lower insertion/removal force and resulting in minimal discomfort to patient. The cuff 16 may be assembled onto the cannula 12 using solvent bond that includes the cuff material. The solvent may also contain a UV-visible compound to ensure glue is present in all locations by illuminating under a UV light. Marking bands, such as UV bands, may be employed to provide specific locations before the cuff 16 is placed onto the cannula 12 to achieve a consistent positioning of the cuff 16 close to the distal tip 38 without occluding the main airway. The cuff assembly may include a step of inverting a distal shoulder 62 of the cuff 16 and keeping an uninverted proximal shoulder 64 to place the cuff 16 close to the distal tip 38 of the cannula 12. Trace amounts of mica may be used on the cuffs to prevent sticking during processing.

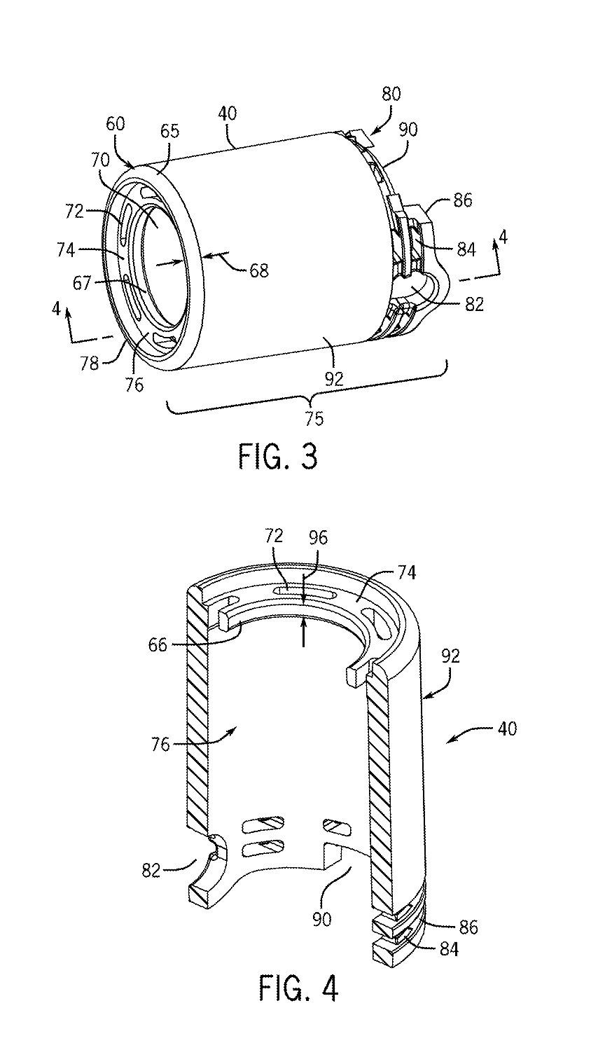

FIG. 3 is a perspective view of the connector cap 40. The connector cap 40 forms a portion of the cannula connector 30 (see FIG. 2). In one embodiment, the connector cap 40 forms a standard 15 mm connector that is permanently affixed to the tracheal tube 10. That is, in contrast to snap-on or removable cannula connectors, the connector cap 40, together with the flange assembly 42 (see FIG. 2) are not removable from each other or from the tracheal tube. The connector cap 40 may be formed by any suitable process, such as molding, e.g., injection molding, or machining. In one embodiment, the connector cap 40 provides dimensional stability and strength for robust connection to a ventilator or breathing device. It is contemplated that, in one embodiment, the connector cap 40 may be provided in a single size that may coupled to flange assemblies 42 and other components of different sizes to provide a standard fitting. In other embodiments, the connector cap may be sized to form specialty fittings.

The connector cap 40 may include an outer lip 65 at a proximal end 60 defining an outer diameter 66 of the connector cap 30 (see FIG. 2) and an inner lip 67 defining an inner diameter 68. The outer diameter 66 may be about 15 mm or 15.5 mm. The inner diameter 68 may be less than 13 mm. For example, the inner diameter 68 may be about 9 mm in one embodiment. The inner diameter defines a portion of the dimensions of the interior passageway 70. The connector cap 40 also includes one or more slots 72 spaced circumferentially about an annular surface 74 formed by the connector cap 40 and recessed relative to the proximal end 60. The slots 72 allow the flow of material in and around the annular surface 74 during manufacturing, as provided herein. While the slots 72 are depicted as generally elliptical, it should be understood that the slots 72 may be any shape and may be present in any number, i.e., one or more. In one embodiment, the slots 72 may be formed or notched out of an exterior surface 92 rather than being disposed on the annular surface 74.

The annular surface 74 may be recessed at least 1 mm relative to the proximal end 60. Further, the annular surface 74 may be recessed relative to the proximal end 60 such that the annular surface is near the proximal end and the position of the annular surface is less than 5 mm from the proximal end 60 or such that the annular surface 74 is positioned at a recess point less than 25% of the total length 75 of the connector cap 40. The annular surface 74 projects (e.g., projects 1-3 mm) from an interior surface 76 of the connector cap to form an abutment angle 78. The abutment angle 78 may be about 90°. In particular embodiments, the abutment angle may be altered to facilitate manufacturing, e.g., flow of overmolded material. In particular embodiments, the annular surface 74 provides additional rigidity and strength to a proximal end 60 of the connector cap 40 by forming a rigid floor under a relatively softer material that forms the flange assembly 42 (see FIG. 2).

The connector cap 40 may also include features at or near a distal end 80 that promote the flow of overmolded material or coupling to other structures. For example, the connector cap 40 may include a passageway 82 to accommodate the inflation line 18 (see FIG. 2). One or more apertures 84 and/or grooves 86 may also facilitate improved coupling to the flange assembly 42 (see FIG. 2). The connector cap 40 may also feature a notch 90 configured to be positioned to align with an inside curve 26 of the cannula 12 when the tracheal tube 10 is assembled (see FIG. 1) and that defines a beveled surface for patient comfort when molded over with the flange assembly 42. After assembly, the interior surface 76 of the connector cap 40 may be coated or covered in a softer material while the exterior surface 92 is exposed. As shown in cross-sectional view on FIG. 4, the annular surface 74 projects from the interior surface 76 of the connector cap 40 but, certain embodiments, is relatively thin. For example, the thickness 96 of the annular surface 74 may be 1-5 mm or less.

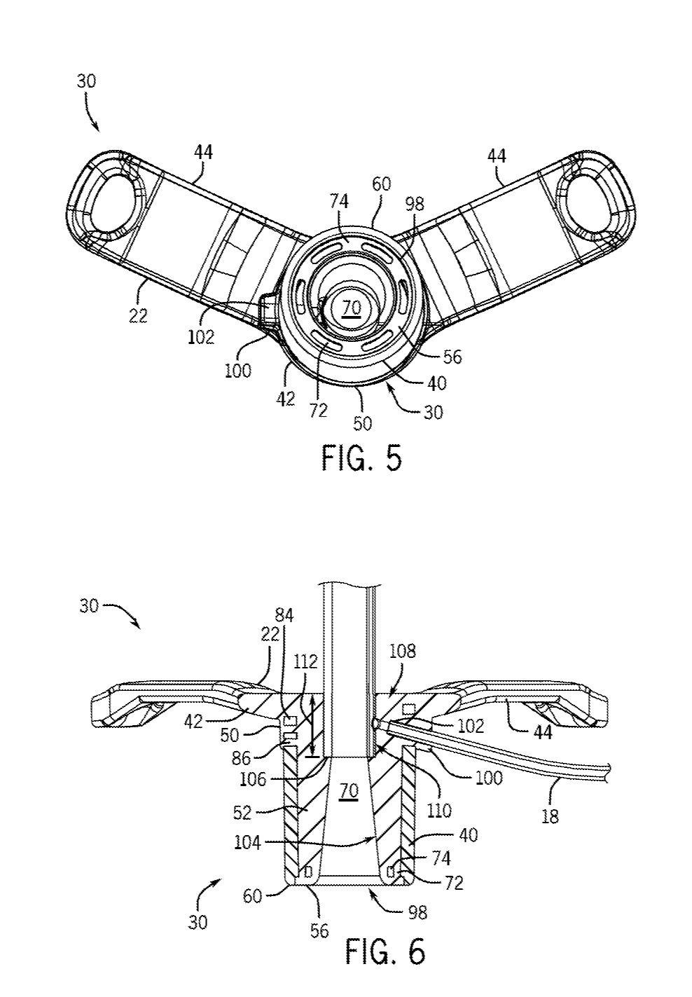

FIG. 5 is a top view of an assembled cannula connector 30. In the depicted embodiment, the material from which the flange assembly 42 is formed is transparent. Accordingly, the slots 72 and the annular surface 74 are visible and are covered by the proximal end 56 of the flange assembly. The proximal end 56 of the flange assembly 42 may align with or be approximately flush with the proximal end 60 of the connector cap 40 to form a relatively smooth tip 98. The cannula connector 30 also includes the flange 22 and extending arm portions 44. The position and/or angle of the extending arm portions 44 may be selected based on the size or use of the tracheal tube 10. For example, neonatal flanges 22 may have wider angle and shorter wings relative to pediatric flanges. The cannula connector 30 may also include a protrusion 100 that features a passageway 102 to accommodate an inflation line 18 (see FIG. 2). The protrusion 100 may be positioned circumferentially about the base 50 of the flange assembly 42 to align with one of the extending arm portions 44 of the flange 22.

In embodiments in which the flange assembly 42 is molded over the connector cap 40, during manufacturing, the material of the flange assembly 42 flows down through the slots 72 during the molding process and covers the annular surface 74 and fills in the slots 72. As shown in FIG. 6, which is a cross-sectional view of the cannula connector 30 coupled to the cannula 12, the interior portion 52 of the flange assembly 42 covers the connector cap 40 and forms an interior surface 104 of the passageway 70 formed by the cannula connector 30 and for transferring ventilator gases. The interior surface 104 may taper from a widest diameter at the tip 98 to narrower diameters closer to the coupling point with a proximal end 106 of the cannula 12. In particular embodiments, the surface 104 may taper to define the passageway as having interior diameters from a largest diameter of about 13 mm to about 9 mm to a narrowest diameter of about 2.5 mm to about 7 mm, which may be selected based on the size of the cannula 12 to be inserted.

The characteristics of the thickness and/or tapering of the interior surface 104 may be defined during manufacturing. As noted, the flange assembly 42 may be molded over the connector cap 40. Such molding may be performed over a single, standard-sized cap 40 to create passageways 110 terminating at the distal end 108 for cannula insertion that are sized to accommodate cannulas of particular sizes. In this manner, only the flange assembly 42 has varying size characteristics while the connector cap 40 component is versatile and may be used for a range of sizes, which may improve manufacturing efficiency. The characteristics of the flange assembly 42 may be defined by the particular size and shape of the mold (not shown) used to form the flange assembly 42 over the connector cap 40. The mold may also define the protrusion 100 and the passageway 102 for the inflation line 18.

The flange assembly 42 is in direct contact with the cannula 12. In particular embodiments, the flange assembly 42 separates the cannula 12 from the connector cap 40 such that the cannula 12 and the connector cap 40 are not in direct contact with one another. A proximal end 106 of the cannula 12 is in contact with and is inserted into a distal end 108 of the base 50 during assembly. The flange assembly 42 forms a passageway 110 configured to accommodate the inserted portion 112 of the cannula. In certain embodiments, the inserted portion 112 is about 7-8 mm. In other embodiments, the inserted portion 112 has a length that is less than half of a total length of the connector cap 40. In yet another embodiment, the passageway 110 is sized such that the distal end 106 terminates at or relatively proximally of the apertures 84 and grooves 86 formed in the connector cap 40. Such an arrangement provides robust coupling of the cannula 12 within the cannula connector 30 without compromising the airway diameter.

In certain embodiments, the passageway 110, prior to insertion of the cannula 12, is larger than an outer diameter of the cannula such that there is clearance between inserted portion 112 and the passageway 110. The clearance may be filled in or sealed during assembly via a suitable adhesive. In this manner, the inserted portion 112, the cannula 12 may maintain its inner diameter along its length. Accordingly, the inner diameter of the passageway 110 may be formed to be slightly larger than the outer diameter of the cannula 12 of the desired size. For example, the passageway 110 may be at least 0.1 mm-3 mm larger than the outer diameter of the cannula 12.

The cannula connector 30 may be coupled to the cannula 12 by any suitable adhesion or bonding process, such as radio frequency (RF) welding. In one embodiment, an inner electrode is placed in the cannula 12 while an outer electrode is placed on the mechanically fit (i.e., molded and formed) flange assembly 42. The passageway 102 for the inflation lumen 18 may be held open mechanically during bonding. For various coupling techniques, there is a risk of the cannula inner diameter being restricted due to dimensional changes in the cannula 12 or connector 30 as a result of the bonding mechanism. In the case of solvent bonding, the materials can swell due to the absorption of the solvent. In the case of RF welding, the heat generated by the welding process may cause a stress relaxation in the cannula 12 and/or the connector 30. In the case of overmolding, the shrinkage of the melted polymer as it solidifies on the cannula may cause the cannula to be compressed. Such effects may be reduced by allowing clearance between the cannula 12 and the passageway 110. That is, the inner diameter of the passageway that mates with the cannula outer diameter may be oversized so that there is clearance between the two components. In addition, in certain embodiments, a piece of tooling, such as a mandrel, may then be inserted into the cannula 12 to expand its outer diameter to expand into the clearance and create an interference between the cannula outer diameter and the passageway 108 inner diameter. In one embodiment, the tooling may have a diameter larger than the cannula inner diameter maximum, e.g., as specified in the International Standards Organization (ISO) standards. The tooling may remain in place as long as necessary for heat or solvent to dissipate and therefore any dimensional changes to have stabilized.

Further, in one embodiment, the flange assembly 42 may be molded over the cannula 12. In such an embodiment, the cannula 12 may be loaded onto a core pin within a mold which will have a diameter larger than the inner diameter maximum. When the melted material is injected into the injection mold, the core pin will counter act the compressive forces of the injection and shrinkage of this material to maintain a desired inner diameter of the cannula 12.

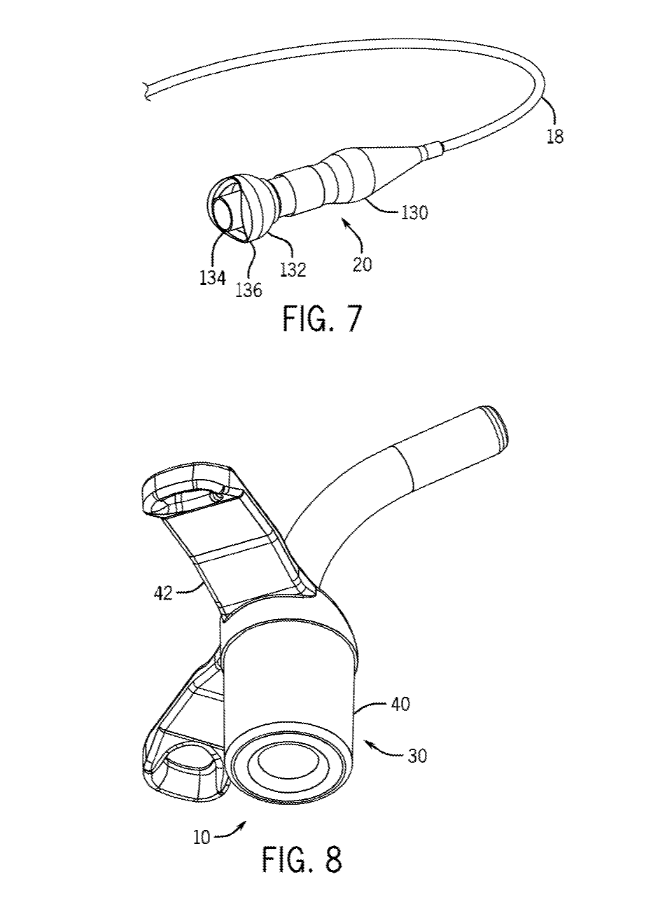

FIG. 7 is a detail view of the pilot balloon assembly 20. The pilot balloon assembly 20 couples to the inflation line 18 to provide an inflation system for the cuff 16 (FIG. 1). The pilot balloon 130 may be dip molded from PVC plastisol. The guard assembly 132 may be injection molded and adhered to the pilot balloon 130 and valve 134 via a solvent bond (cyclohexanone). The guard 132 may prevent an operator from confusing the valve 134 with other components, e.g., using the inflation line 18 as a suction line. Further, the guard 132 may prevent the pilot balloon assembly 20 from being stored within the passage 70 when the tracheal tube 10 is not in use, which may preserve the integrity of the pilot balloon 130. That is, the guard 132 may provide a physical obstruction to prevent improper storage. As shown, the guard 132 may incorporate opposing wings 136. However, other structures are also contemplated, such as annular structures, disc structures, and so forth. The pilot balloon 130 may be a pressure-limiting pilot balloon or include pressure-limiting features.

FIG. 8 is a perspective view of an alternative tracheal tube 10 that is cuffless. In the depicted embodiment, the features of the cannula connector 30 may include a connector cap 42 and flange assembly 42. However, the cannula connector 30 does not include any passageways to accommodate an inflation line. In certain embodiments, the connector cap 40 that is used for the cuffed embodiment may also be used to form a cuffless cannula connector 30. In such an embodiment, any holes for the inflation assembly are molded over with a suitable mold or mold form. In other embodiments, a cuffless connector cap without an inflation line passage (e.g., without passage 82, FIG. 3) Further, the overmolding and cannula coupling may be accomplished in a manner similar to the cuffed embodiment as provided herein, but without accommodations for holding an inflation line passageway open.

Components of the tube assembly 10 may be manufactured according to suitable techniques, such as those provided herein. For example, the cannula 12 and the cannula connector 30 may be molded, overmolded, two shot molded, computer numerical control (CNC) machined, milled, or otherwise formed into the desired shape. In one embodiment, a mold or mold form may be used to manufacture components of the tracheal tube 10. One or more components may be manufactured of materials such as a polyethylene (e.g., low density polyethylene), polypropylene, PTFE, expandable PTFE, polyvinyl chloride (PVC), a PEBAX silicone, a polyurethane, thermoplastic elastomers, a polycarbonate plastic, a silicon, or an acrylonitrile butadiene styrene (ABS). In particular embodiments, the tracheal tube 10 may be formed from phthalate-free materials, e.g., all or a portion of the individual components of the tracheal tube 10 are phthalate-free.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to airway devices, but these techniques may also be utilized for connections between inner and outer conduits for other types of medical devices and medical connective tubing. Rather, the various embodiments may cover all modifications, equivalents, and alternatives falling within the spirit and scope of the disclosure as defined by the following appended claims.

What is claimed is:

1. A tracheal tube assembly comprising:
a cannula configured to be positioned in a patient airway; and
a cannula connector comprising:
a cannula connector cap comprising a plurality of slots spaced about the cannula connector cap at or near a proximal end of the cannula connector cap; and
a flange assembly coupled to the cannula connector cap and the cannula and comprising a flange, wherein a portion of the flange assembly extends through the slots.

2. The tracheal tube assembly of claim 1, wherein the plurality of slots are disposed on a surface recessed relative to a proximal end of the cannula connector cap.

3. The tracheal tube assembly of claim 2, wherein the surface projects towards an interior of the cannula connector cap and the surface comprises an annulus.

4. The tracheal tube assembly of claim 2, wherein the surface forms about a 90° angle with an interior surface of the cannula connector cap.

5. The tracheal tube assembly of claim 1, wherein the portion of the flange assembly is disposed on an interior of and coaxially with the cannula connector cap to define a passageway formed by the cannula connector.

6. The tracheal tube assembly of claim 1, comprising a passageway formed in the flange assembly and extending through the cannula connector cap to an exterior surface of the cannula connector.

7. The tracheal tube assembly of claim 6, comprising an inflation line disposed in the passageway and coupled to an inflatable cuff disposed on the cannula.

8. The tracheal tube assembly of claim 1, wherein the portion of the flange assembly that extends through the slots terminates at a position that is approximately flush with a proximal end of the cannula connector cap.

9. The tracheal tube assembly of claim 1, wherein the cannula connector cap comprises one or passageways at or near a distal end and wherein the flange assembly extends through the one or more passageways to couple to extending arm portions of the flange.

10. The tracheal tube assembly of claim 9, wherein the one or more passageways are disposed within a groove recessed relative to an exterior surface of the cannula connector cap.

11. The tracheal tube assembly of claim 1, wherein a distal end of the cannula connector cap comprises a notch that aligns with a beveled portion of the flange assembly and wherein the notch is disposed about 90° from one or both arms of the flange.

12. The tracheal tube assembly of claim 1, wherein the inner diameter of the cannula connector tapers towards the cannula.

13. The tracheal tube assembly of claim 1, wherein a proximal end of the cannula is coupled via adhesion or bonding to the flange assembly such that the cannula does not directly contact the cannula connector cap.

14. The tracheal tube assembly of claim 1, wherein flange assembly is formed from a material that is at least partially transparent.

15. A method of manufacturing a tracheal tube assembly comprising:
providing a cannula connector cap comprising a plurality of slots spaced about a circumference of the cannula connector cap at or near a proximal end of the cannula connector cap;
molding a flange assembly to form a flange coupled to the cannula connector cap, wherein molding the flange assembly comprises molding over at least a portion of the cannula connector cap such that a portion of the flange assembly extends through the slots and wherein the flange assembly is disposed inside of the cannula connector cap to define an interior passageway; and
coupling a proximal end of a cannula to the interior passageway.

16. The method of claim 15, wherein coupling the proximal end of the cannula to the interior passageway comprises RF welding, wherein a first electrode is applied to an interior of the cannula and wherein a second electrode is applied to the flange assembly.

17. The method of claim 15, wherein coupling the proximal end of the cannula to the interior passageway comprises overmolding the proximal end of the cannula and wherein the cannula is loaded onto an oversized core pin.

18. The method of claim 15, wherein coupling the proximal end of the cannula to the interior passageway comprises solvent bonding the proximal end of the cannula, and wherein the proximal end of the cannula is widened with an oversized mandrel.

19. The method of claim 15, wherein molding the flange assembly comprises molding an exit port configured to accommodate an inflation line about a passageway formed in the cannula connector cap.

20. The method of claim 15, wherein the interior passageway is wider than an outer diameter of the cannula at a distal end of the interior passageway such that there is clearance between the cannula and the interior passageway before the coupling.

21. The method of claim 15, comprising inserting a tool into the inner cannula to widen the proximal end before the coupling and removing the tool after the coupling is complete.

22. A tracheal tube assembly comprising:
a cannula configured to be positioned in a patient airway;
an inflatable cuff disposed on the cannula;
a cannula connector cap;
a flange assembly molded over a portion of the cannula connector cap to form an interior passageway of the cannula connector cap and coupled to the cannula;
an inflation line in fluid communication with the inflatable cuff and extending through the flange assembly and the cannula connector cap, wherein the inflation line terminates in a pilot balloon assembly and wherein the pilot balloon assembly comprises opposing wings that surround a valve configured to receive an inflation device:

23. The tracheal tube assembly of claim 22, wherein the inflatable cuff comprises mica powder.

24. The tracheal tube assembly of claim 22, comprising a protrusion formed in the flange assembly proximal of the flange and comprising an exit port for the inflation line.
